(19) United States
(12) Patent Application Publication
(10) Pub. No.: US 2012/0042737 A1
(43) Pub. Date: Feb. 23, 2012

(54) NOVEL DISSOLUTION TESTING SYSTEM AND APPARATUS WITH OFF-CENTER IMPELLER

(75) Inventors: Piero M. Armenante, Millington, NJ (US); Yimin Wang, North Arlington, NJ (US)

(73) Assignee: NEW JERSEY INSTITUTE OF TECHNOLOGY, Newark, NJ (US)

(21) Appl. No.: 13/086,382

(22) Filed: Apr. 13, 2011

Related U.S. Application Data
(60) Provisional application No. 61/323,419, filed on Apr. 13, 2010.

(51) Int. Cl. G01N 33/00

(52) U.S. Cl. 73/866

(57) ABSTRACT

In the pharmaceutical industry, dissolution testing is a critical step in quality control and a standard method for assessing batch-to-batch consistency of solid oral drug delivery systems, such as tablets. One of the most widely used dissolution test devices is the UPS Apparatus 2 (paddle). At present, dissolution testing remains susceptible to significant error and test failures. Previous studies indicate that poor reproducibility of dissolution testing data and inconsistency of dissolution results can arise from the complex hydrodynamics present in the unbaffled, hemispherical-bottom, agitated vessel that constitute the UPS Apparatus 2. In the present invention, a novel dissolution testing apparatus was constructed in which the impeller was placed off-center with respect to the center point of the vessel bottom. It has been shown that the dissolution profiles in the present invention were not significantly affected by tablet location as confirmed by the value of the factors f₁ and f₂, which were well within the accepted ranges and did not change appreciably with the tablet location. By contrast, the corresponding dissolution tests for current systems failed these similarity tests. In addition, the flow fields near the vessel bottom were obtained via CFD simulation and were found to be significantly more uniform in e present invention than in the current standard apparatus. The present invention has the potential of becoming a valid alternative to the standard USP dissolution testing apparatuses used for dissolution testing.

### CROSS-REFERENCE TO RELATED APPLICATION(S)

This application claims the benefit of U.S. Provisional Patent Application No. 61/323,419 filed Apr. 13, 2010, the entirety of which is incorporated herein by reference.

### FIELD OF THE INVENTION

This invention relates to the field of dissolution testing.

### BACKGROUND OF THE INVENTION

Dissolution testing is routinely carried out in the pharmaceutical industry to determine the rate of dissolution of solid dosage forms. In addition to being routinely used by pharmaceutical companies to demonstrate adequate drug release in vivo, in vitro dissolution testing is used to assist with formulation design, process development, and especially the demonstration of batch-to-batch reproducibility in production. Dissolution testing is one of several tests that pharmaceutical companies typically conduct on oral dosage formulations (e.g., tablets, capsules, ect.) to determine compliance and to release products for distribution and sales.

Thus, this method is routinely used in the pharmaceutical and biotechnology industry to formulate drug dosage forms and to develop quality control specifications for its manufacturing processes. Although dissolution appears to be a simple process, developing a suitable dissolution test requires careful consideration of operation variables such as the agitation speed, temperature control, dissolution medium, dosage form designs and other important variables.

Although the United States Pharmacopoeia & National Formulary (USP) lists several different dissolution test apparatuses, most dissolution tests are currently conducted with USP Dissolution Test Apparatuses 1 and 2. The USP Dissolution Test Apparatus 2 is the most commonly and widely used apparatus specified by USP. The dimensions, characteristics, and operating conditions of USP Dissolution Test Apparatus 2 are detailed by USP, and most users typically conform to these conditions when conducting dissolution tests. United States Pharmacopeia 31/National Formulary 26. 2008.

The value of in vitro dissolution testing as a quality control tool is demonstrated by its long history of regulatory acceptance. It has been included in the USP since 1968 and continues to be an important test today. This kind of test fulfills a regulatory requirement. Although the primary purpose of the dissolution test specification is to distinguish between acceptable and unacceptable batches, it is also used as a measure of batch-to-batch consistency of the manufacturing process. Most solid oral dosage forms are required to have a dissolution test, and it is not uncommon to have a drug recall due to a failed dissolution test. In an effort to ensure that the drugs which have the same properties can be produced all the time, pharmaceutical scientists have utilized in vitro dissolution testing as a quality control tool for formulation development, manufacturing process assessment, and prediction of bioequivalence of drug. The batch to batch quality of a product is often determined by conducting dissolution tests with predefined procedures by USP.

During the drug approval process, pharmaceutical companies are required to submit to the FDA in vitro dissolution data and related bioavailability results. Before a drug product is approved for use in humans, dissolution specifications are established to ensure that all batches produced are bioequivalent. The final dissolution specifications of a new product are then published in the USP as compendial standards as they will become the official specifications for subsequent products containing the same active ingredients.

Solid dosage forms, such as tablets, are a convenient way of administering drugs to patients. Upon ingestion, tablets disintegrate into smaller fragments in the body compartment where absorption by the body is initiated, typically in the stomach or the upper intestine. These fragments dissolve in the digestive juices and can become absorbed by an epithelial layer such as the lining of the upper intestine. This complex in vivo process is routinely simulated by in vitro dissolution tests mandated by the Food and Drug Administration (FDA) and specified in USP. The present invention relates to a novel apparatus to conduct in vitro dissolution testing of solid dosage forms.

A review of the literature shows that there have been numerous reports describing high variability of test results, even for dissolution apparatus calibrator tablets. See D. Cox & W. Furman, J. Pharma. Sci., 71 (1982) 451-452; T. Moore et al., Pharmacopeial Forum, 21 (1995) 1387-1396; S. Qureshi & J. Shabnam, Eur. J. Pharm. Sci., 12 (2001) 271-276. Furthermore, the hydrodynamics of USP apparatus 2 appears to play a major role in the poor reproducibility of dissolution testing data and the inconsistency of dissolution results. This is not surprising considering that the USP Apparatus 2 is a small, unbaffled vessel with a hemispherical bottom provided with a slowly rotating paddle, in which a tablet (or another dosage form) is dropped. As it has been known for decades to reaction engineers, such complex hydrodynamics in such a small vessel would have a direct impact on mass transfer rates and, consequently, on dissolution rate. Furthermore, the tablet dissolution process is intrinsically complex since it involves solid-liquid mass transfer, particle erosion, possible disintegration, particle suspension and particle-liquid interactions.

This process is further complicated by the interactions of the complex tri-dimensional flow with the dissolving tablet and its fragmented particles, the highly variable velocity, energy and shear stress distribution as a function of tablet location within the Apparatus, and the uncertainty in the location of the tablet upon its release inside the apparatus. Literature reports confirm these observations and the potentially important role of hydrodynamics on the dissolution process and the inconsistency of dissolution test results. See D. Cox & W. Furman, J. Pharma. Sci., 71 (1982) 451-452; T. Moore et al., Pharmacopeial Forum, 21 (1995) 1387-1396; S. Qureshi & J. Shabnam, Eur. J. Pharm. Sci., 12 (2001) 271-276; J. Mauger et al., Dissolution Tech., 10 (2003) 6-15; S. Quresi & I. McGilveray, Eur. K. Pharm. Sci., 7 (1999) 249-258; P. Costa & J. Lobo, Drug Dev. Industry Pharm., 27 (1990) 811-817; L. Bocanegra et al., Drug Dev. Industrial Pharm., 16 (1990) 1441-1464; D. Cox et al., J. Pharm. Sci., 72 (1983) 910-913; G. Bai, et al., Int. J. Pharmaceutics, 403 (2011) 1-14.

Over the years, much of the intrinsic variability of the test has been recognized and attempted to be addressed by using calibrator tablets to try to quantify it. The concept is simple: If highly uniform product gives variable results, this degree of variability can be attributed to the method and therefore subtracted from the variability observed in the product.

Knowledge of operating and geometry variables for a dissolution apparatus is important to the pharmaceutical scientist who is interested in product development and quality control. The ability of a dissolution test to show changes in so many parameters is its power and its frustration. The hydrodynamics within USP Apparatus 2 is highly non-uniform which may yield substantial variability in dissolution rate measurements. It is believed that the poorly reproducible and inconsistent results stem from the complicated hydrodynamics with the apparatus. The importance of such is highlighted by the fact that failed dissolution tests resulted in 47 products recalls in 2000-2002, representing 16% of non-manufacturing recalls for oral solid dosage forms.

Failed dissolution tests can result in product recalls, costly investigations, and potential production delays, all of which have a substantial financial impact on the pharmaceutical industry. These inconsistencies present even greater challenges when trying to implement quality by design (QbD), which defines the future state of dissolution, its value, method design, and links to the design space. Scientists and researchers have investigated different operation and other variables to address the non-reproducibility issue of dissolution testing.

Due to the urgent call for a more reliable USP Apparatus 2 from regulatory agents and the pharmaceutical industry the present invention embraces a device that would avoid the defects that arise from the poor hydrodynamics of current systems. The new dissolution system would utilize engineering principles and methods to design a test where the flow is relatively homogeneous and does not introduce undue variability while also retaining the existing basic equipment and the knowledge that operators have accumulated over the years.

### SUMMARY OF THE INVENTION

Even though calibrator tablets are now used in dissolution testing, the variability of results remains an issue. The release profiles from the current Dissolution Apparatus 2 do not always lead to a reproducible pattern. One main reason that caused the non-repeatable release profile is the location of the drug inside the vessel G. Bai & P. Armenante, J. Pharm. Sci., 98 (2009) 1511-1531. Since dosage forms are typically manually dropped into the agitated vessel at the beginning of each test, said form can be randomly located on the bottom of the vessel. The eventual location of the dosage form seems to have a significant effect on the release profile under the current system. One goal of the present invention is to obtain a more robust system wherein the release profile is not affected by the location of dosage forms.

Dissolution testing for control purposes with respect to multiple embodiments the present invention was conducted on a Distek Premiere 5100 Bathless Dissolution System (Distek Inc., North Brunswick, N.J.). There are seven built-in vessel positions in this particular dissolution system. The volume of each vessel has a range of 400 ml-1000 ml, which is programmable by setting parameters in the LED Display. The RPM can be digitally controlled from 25 rpm to 300 rpm with a resolution of 0.1 rpm. The accuracy of the RPM control is +/−0.2 rpm. The temperature of each individual vessel is continuously monitored and controlled with a resolution of 0.01° C. The accuracy of temperature control is +/−0.25° C. The shafts wobble is controlled less than 0.010″ TIR. All the settings can be changed by pressing up, down, left, right buttons on the 0.75 inch high output four-digit LED display screen. This dissolution system is representative of many of the dissolution systems on the market today. The present invention can be utilized with any similar such bathless or water bath containing apparatus and said apparatus is used for illustrative purposes only.

For multiple embodiments of the present invention the agitating impeller and shaft are placed off-center with respect to the center point of the dissolution testing vessel. In such an asymmetric system, a ‘baffle effect’ is introduced into the flow. This non-intrusive method successfully induces a baffle effect without a physical baffle having to be placed in the vessel. This allows testing to be done with minimal change to the current system while still obtaining a much more robust testing environment. This is because the flow inside the vessel of certain exemplary embodiments of the present invention are no longer symmetric but more uniform near the vessel bottom, where the oral dosage form is located. This causes the release profiles produced by said embodiments to be reproducible irrespective of table location at the bottom of the vessel.

Embodiments of the present invention embrace a multitude of methods for achieving an off-center position between an impeller and a vessel bottom. The present invention embraces any method of shifting the impeller off-center with respect to the vessel bottom because said invention embraces being utilized with current devices as well as part of newly manufactured devices so as to allow for ease of adoption within the dissolution testing community. The exemplary embodiments herein are meant to merely illustrate certain technical possibilities to achieve said shift.

The present invention embraces embodiments utilizing a dissolution medium volume of about 300 ml to about 1000 ml.

The impeller agitation speed for certain embodiments of the present invention is between about 25 rpm and about 200 rpm.

In one preferred embodiment of the present invention agitation speed is between about 35 rpm and about 100 rpm.

Embodiments of the present invention embrace a dissolution temperature of about 20° C. and about 37° C.

In one preferred embodiment of the present invention the temperature for dissolution testing was set to about 37° C.

The present invention embraces a shift of impeller with respect to the vessel in the range of about 0.1 mm to about 13 mm.

In a preferred embodiment of the present invention the impeller is offset about 8 mm from center of vessel.

For certain embodiments of the present invention, the vessel body itself is shifted to achieve an off-center impeller configuration while in other embodiments the impeller and shaft are shifted to achieve said configuration.

### BRIEF DESCRIPTIONS OF THE DRAWINGS

The teachings of the present invention can be readily understood by considering the following detailed description in conjunction with the accompanying drawings, in which:

FIG. 1A displays a side view of a version of the current USP 2 Apparatus with measurements;

FIG. 1B displays a side view of a version of the current USP 2 Apparatus;

FIG. 1C displays a side view of one embodiment of the present invention;

FIG. 1D displays a side view of one embodiment of the present invention;

FIG. 2 displays a top view of a vessel cavity, a top view of vessel cavity with vessel, and a top view of a vessel holding plate;

FIG. 3A is a top view of a symmetric insert;

FIG. 3B is a top view of an asymmetric insert;

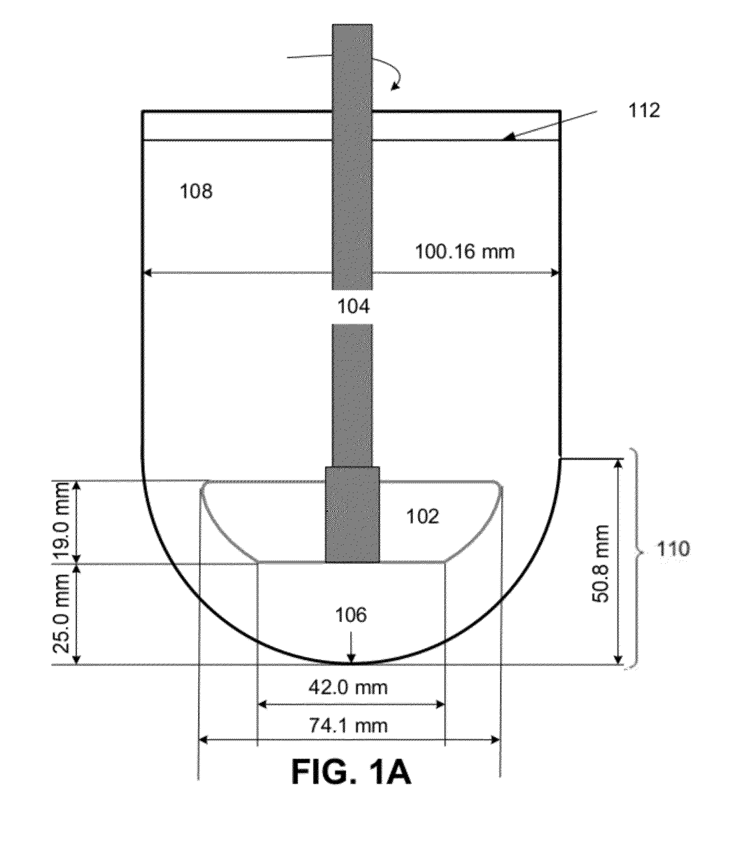

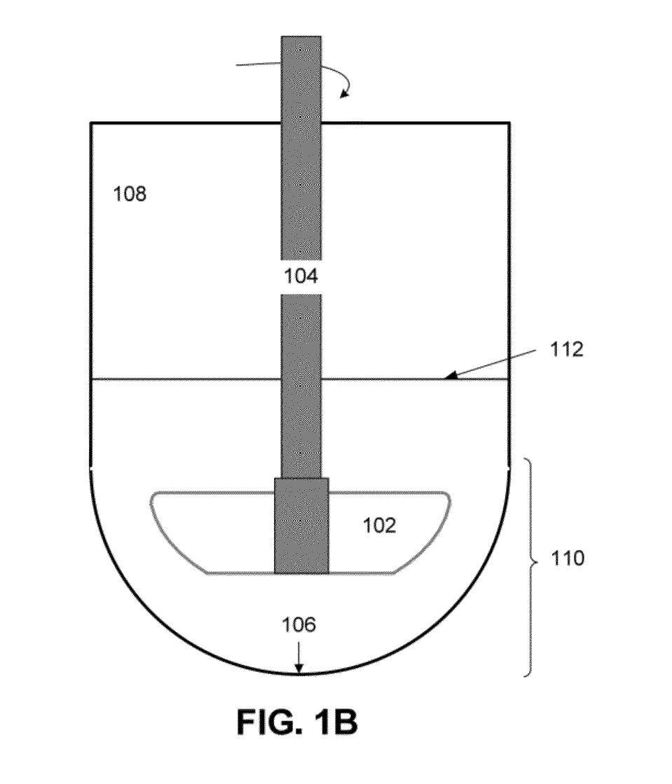

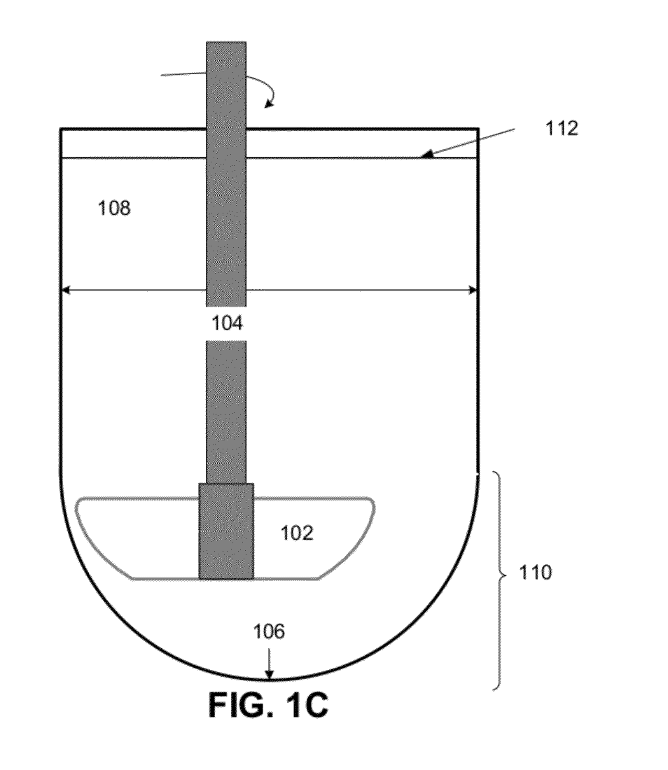

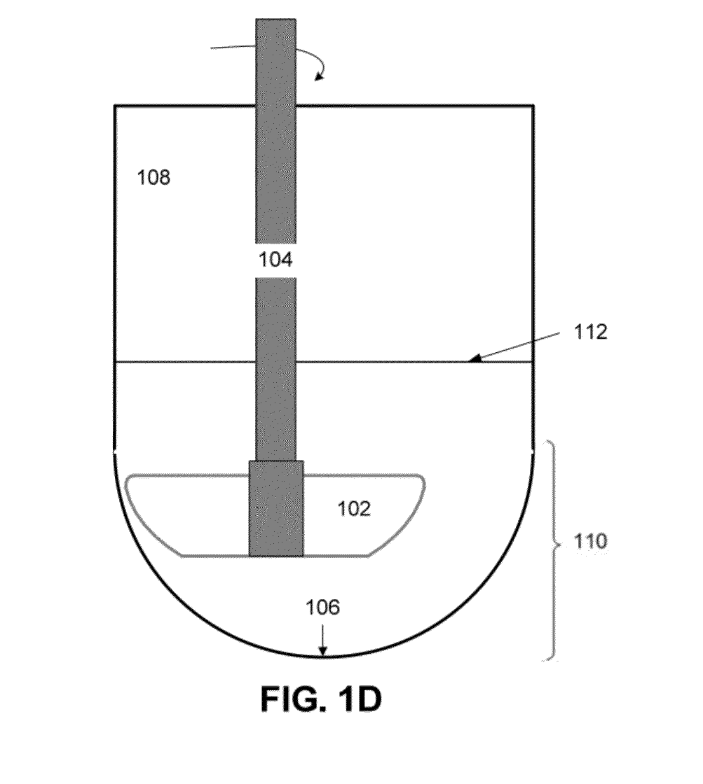

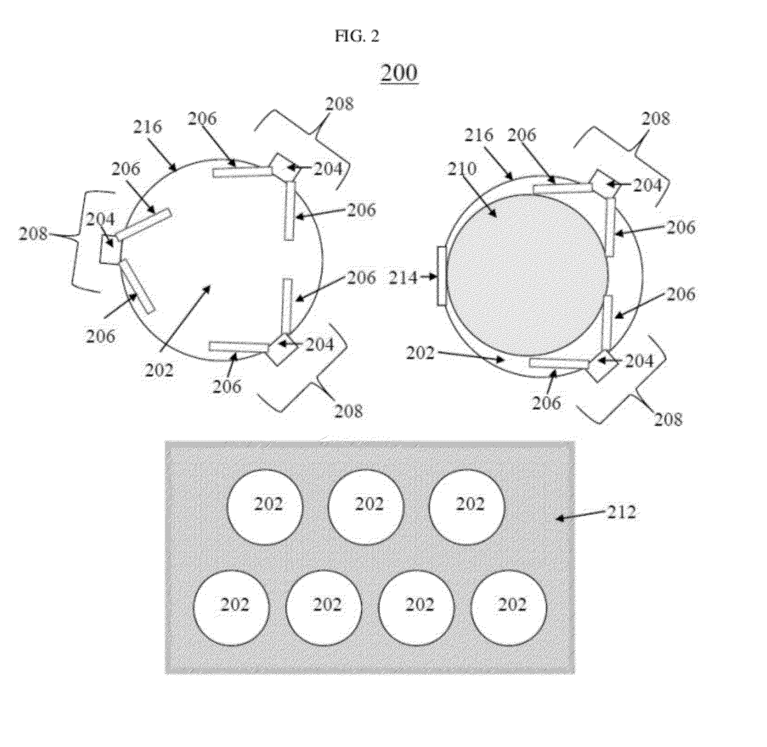

FIG. 1C is side view of a symmetric insert;

FIG. 1D is a view of an asymmetric insert;

FIG. 4 is a top view of a vessel holding plate and lever system;

FIG. 5 is a top view of a motor holding assembly and lever system;

FIG. 6 is a is a graphical depiction of a calibration curve of prednisone tablets;

FIG. 7 is a is a graphical depiction of a calibration curve of salicylic acid tablets;

FIG. 8 is a graphical depiction of dissolution results for prednisone tablets using Apparatus 2;

FIG. 9 is graphical depiction of dissolution results for prednisone tablets in one embodiment of the present invention;

FIG. 10 is a graphical depiction of dissolution results for salicylic acid tablets using USP Apparatus 2;

FIG. 11 is a graphical depiction showing dissolution results for salicylic acid tablets in one embodiment of the present invention; and

FIG. 12 displays location of tablets on vessel bottom as seen in Tables 2 & 3.

To facilitate understanding, identical reference numerals have been used, where possible, to designate identical elements that are common to the figures.

### DETAILED DESCRIPTION OF THE INVENTION

The following is a detailed description of the invention provided to aid those skilled in the art in practicing the present invention. Those of ordinary skill in the art may make modifications and variations in the embodiments described herein without departing from the spirit or scope of the present invention. Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention. All publications, patent applications, patents, figures and other references mentioned within this document are expressly incorporated by reference in their entirety.

Referring to FIG. 1, for multiple embodiments of the present invention an impeller 102 and a shaft 104 is placed off-center with respect to the center point 106 of a hemispheric vessel 108 bottom 110 with an air-liquid interface 112. In such an asymmetric system, a ‘baffle effect’ is introduced into the flow. This non-intrusive method successfully induces a baffle effect without a physical baffle having to be placed in the vessel. The flow inside the vessel of the present embodiment is no longer symmetric but more uniform near the vessel bottom, where the oral dosage form is located. This causes the release profiles produced by certain exemplary embodiments of the present invention to be reproducible irrespective of table location at the bottom of the vessel.

Further embodiments of the invention described below indicate alternative methods for achieving an off-center position between impeller 102 and vessel bottom 110. These further embodiments are meant to merely illustrate certain technical possibilities to achieve said shift. Said embodiments are by no means exhaustive, merely illustrative, in fact the present invention embraces all reasonable methods for securing and shifting a vessel device within a vessel cavity.

In certain embodiments of the present invention the volume of each vessel 108 is about 500 ml FIG. 1C and about 900 ml FIG. 1D.

The impeller 102 agitation speed for certain embodiments of the present invention is between about 25 rpm and about 200 rpm. In one preferred embodiment of the present invention agitation speed is between about 35 rpm and about 100 rpm. For each impeller speed currently associated via USP classification for any dosage form, embodiments of the inventive system can be tested to obtain the agitation speed that will provide a dissolution profile that mirrors that of current classification standards so that the same dissolution profile can be obtained with the present invention as with current, USP approved dissolution testing systems. Consequently, one embodiment of the present invention operates at an agitation speed of 35 rpm which mimics current USP classification system data for dissolution tests occurring at 50 rpm. A further embodiment of the present invention has an agitation speed of 66 rpm which mimics current USP classification system data for dissolution tests occurring at 100 rpm.

The temperature for certain embodiments of the present invention is between about 20° C. and about 37° C. In one preferred embodiment of the present invention the temperature for dissolution testing was set to about 37° C.

In embodiments of the present dissolution system, the inventive system is modified from a common dissolution system, with said embodiments utilizing as a model the Distek 5100 Bathless Dissolution Apparatus 2 (Distek Inc., North Brunswick, N.J.). However, this system is used as a model and all exemplary embodiments of the present invention could be practiced with any other similar current and/or newly developed dissolution system of the same general type.

In multiple embodiments of the present invention the position of impeller 102 and shaft 104 is changed to achieve an off-center configuration. The shift of impeller with respect to the vessel 108 is from about 0.1 mm to about 13 mm. Said range is meant to embrace any shift from slightly off-center to the point where impeller 102 is located as close as possible to the vessel edge without making contact, thus maximizing baffle effect. In a preferred embodiment of the present invention the impeller is offset about 8 mm from center of vessel.

In further embodiments of the present invention, instead of changing the position of impeller 102 and shaft 104, the vessel body itself is shifted to achieve an off-center impeller configuration.

Referring to FIG. 2, illustrating one embodiment implementing the vessel body shift configuration, each vessel cavity 202 is fitted with a plurality of removable spring 206 and screw 204 assemblies 208. The screws are loosened in order to relocate the vessel body 210 within a vessel cavity 202. In certain embodiments of the present invention a plurality of vessel cavities are found within a vessel holding plate 212. One or more assemblies 208 are removed and replaced with a pad 214. The screws 206 of the remaining assemblies are tightened uniformly until the vessel bottom 110 and impeller 102 (not shown in FIG. 2) are located in their desired off-center configuration. While FIG. 2, illustrates a system utilizing three assemblies 208 the present invention embraces all potential number of combinations of assemblies 208 and pads 214 to create the desired off-center effect. Further embraced but not shown, are gaskets and/or support materials placed around the vessel to stabilize the new position. The screw 204 is actually a flexible piece of rubber, molded plastic, or other like material that holds the vessel in place by using the tension created by tightening of the screw 204.

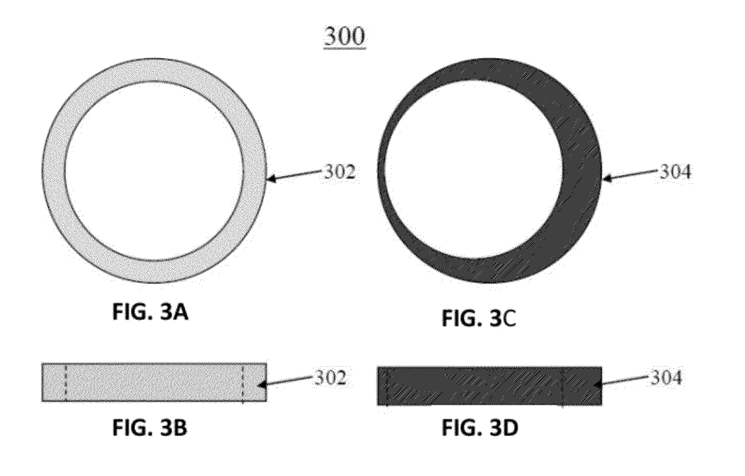

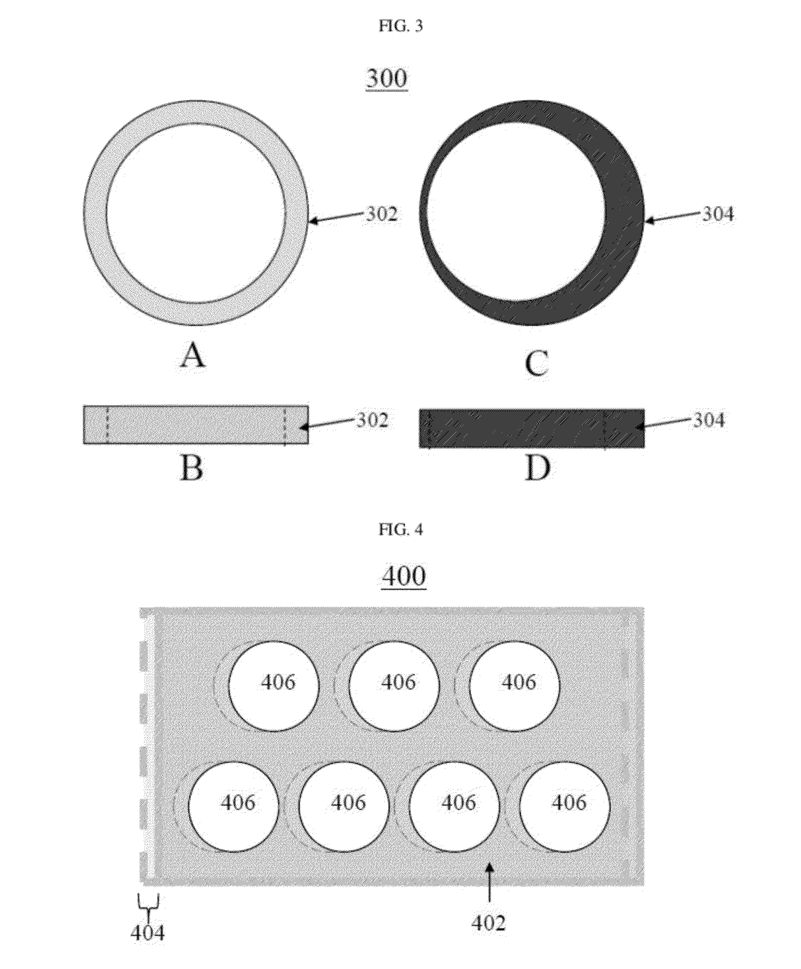

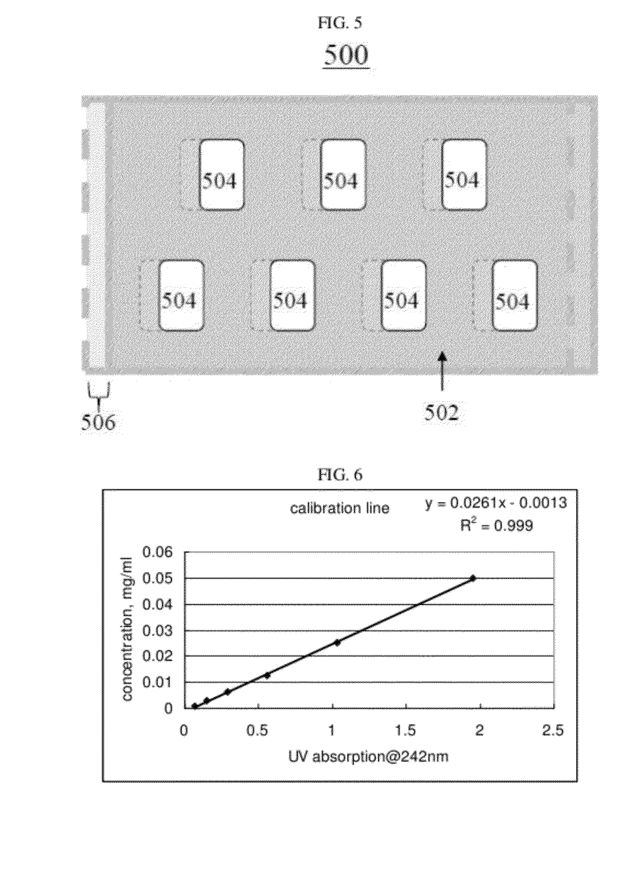

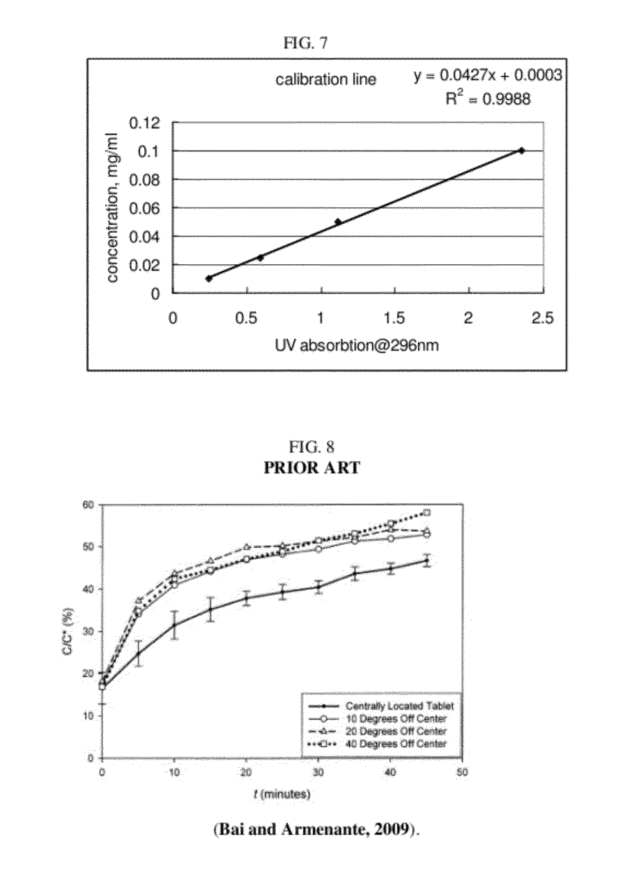

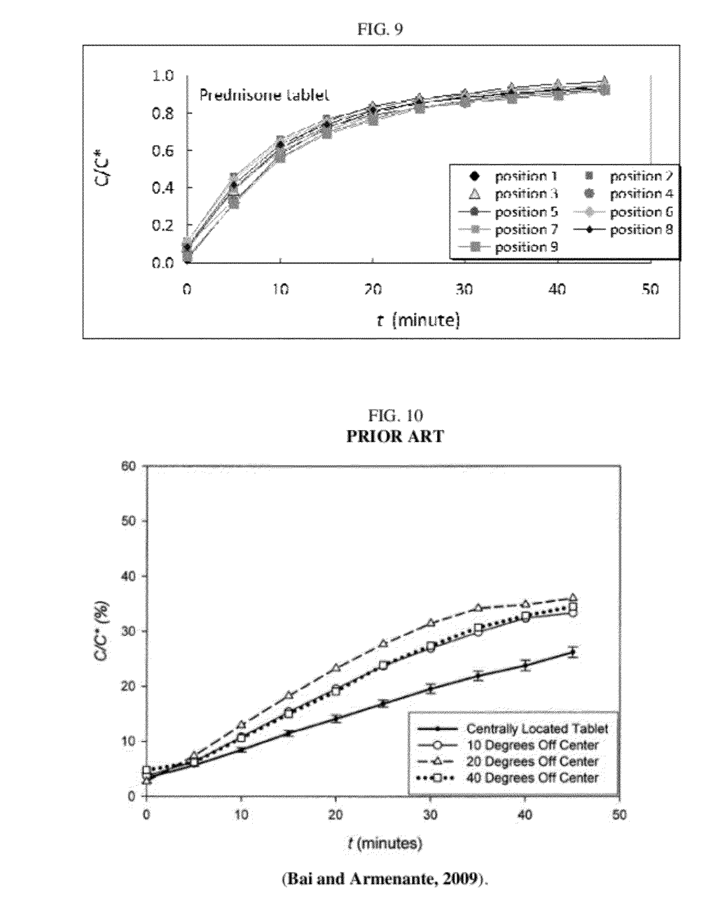

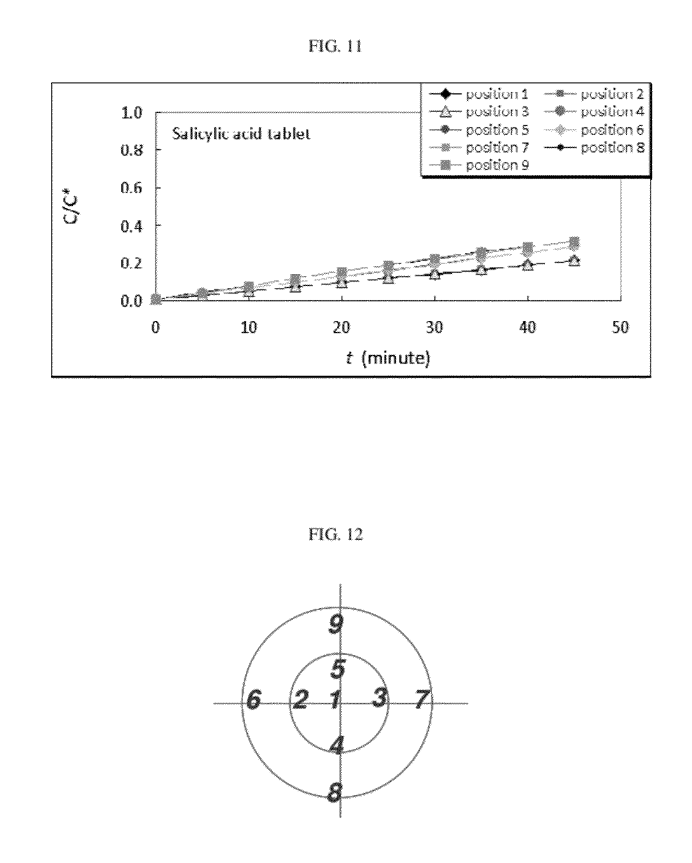

FIG. 3 illustrates one aspect present invention practicing a shift in position of a vessel bottom 110 with respect to an impeller 102 shown in FIG. 1 (not shown in FIG. 3) is placed, by means of an asymmetrical insert (FIG. 3 C-D) 304 within the top lip 216 of the cavity 202 shown in FIG. 2 (not shown in FIG. 3) of the dissolution system where a vessel 210 shown in FIG. 2 (not shown in FIG. 3) is placed. Materials to be utilized for insert construction include but are not limited to molded plastic or rubber. Said insert 304 could be part of a color coded system in which one molded insert aligns a vessel for standard USP protocol (FIG. 3 A-B) 302 while the second molded insert 304 of a different color than the first 302 would align a vessel in an off-center fashion as described herein. There could also be a series of asymmetrical inserts 304 of varying thickness to achieve various distances off-center with respect to vessel bottom 110 and impeller 102 shown in FIG. 1 (not shown in FIG. 3) from about 0.1 mm to about 13 mm.

Referring to FIG. 4, in further embodiments of the present invention, a vessel cavity shift is achieved by utilizing a vessel holding plate 402 in which said plate is connected to a lever (not shown) which, when engaged, causes a shift in vessel placement 404 allowing for a shifted vessel cavity position 406 relative to original position. The present invention embraces a holding plate 402 with multiple settings allowing for multiple shifts in distance such that the one could achieve various, metered, predetermined distances off-center with respect to the vessel cavity from original position to shifted position 406 from about 0.1 mm to about 13 mm to allow for off-center impeller placement from a dissolution testing system as seen in previous embodiments of the present invention.

Referring to FIG. 5, in one further exemplary embodiment of the present invention the impeller shift was achieved by utilizing a motor holding assembly 502 where the motor drives 504 are attached, in which the assembly is connected to a lever (not shown) which, when engaged, causes a shift in motor drive placement 506 allowing for an off-center placement of the vessels relative to an impeller 102 FIG. 1 (not shown in FIG. 5). The present invention embraces a motor holding assembly 502 with multiple settings allowing for multiple shifts in distance such that the one could achieve various, metered, predetermined distances off-center with respect to the vessel cavity from original position to shifted position 506 from about 0.1 mm to about 13 mm to allow for off-center impeller placement from a dissolution testing system as seen in previous embodiments of the present invention.

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed

Certain embodiments of the present invention were utilized for experimentation to prove the robustness of said invention (Table 1). In one such test, the dissolution medium for one exemplary embodiment of the present invention utilizing prednisone calibrator tablets, a representative of a dissolving dosage form and as a test drug of choice was distilled water, which was de-aerated follow USP recommended procedures. The dissolution medium for an exemplary embodiment of the present invention utilizing salicylic acid tablet, a representative of a non dissolving dosage form was prepared.

TABLE 1
Detailed operation conditions of dissolution tests

| | Prednisone tablet | Salicylic acid tablet |
|---|---|---|
| Dose | 10 mg | 300 mg |
| Medium | 500 ml | 900 ml |
| | de-aerated, de-ionized | pH = 7.4 buffer |
| | water | (KH₂PO₄ + NaOH) |
| Temperature | 37° C. | 37° C. |
| Agitation speed | 50 rpm | 100 rpm |
| Filter | PVDF 0.45 um | PVDF 0.45 um |
| UV wavelength | 242 nm | 296 nm |
| Standard solution | NCDA dissolution test | USP Salicylic Acid |
| | performance standard | Tablets RS Lot Q0D200 |
| Time | 5-min interval; 45 min | 5-min interval; 45 min |
| | total | total |

The procedure for the examples described in the paragraph above is loosely based on the USP 2006. United States Pharmacopeia 31/National Formulary 26. 2008. General Chapter <711> Dissolution. 12601. Temperature during all the dissolution testing for said examples was about 37° C. Dissolution medium was heated to about 37° C. prior to its use. In order to test the effect of tablet position during dissolution test, and robustness of the current testing approach along with certain embodiments of the present invention a dosage form for each tablet was placed at nine predefined spots on the vessel bottom 210 with a very small bead of commercial glue. Once the tablet and the vessel are setup properly, the pre-prepared dissolution medium (500 ml for prednisone tablet, 900 ml for salicylic acid tablet) was gently poured into the vessel as seen in FIG. 1a-b.

In embodiments tested experimentally, agitation commenced immediately after the addition of dissolution medium. The agitation speed is 50 rpm for the disintegrating dosage form example, prednisone, tablet and 100 rpm for the non-disintegrating dosage form example, salicylic acid tablet as per USP protocol. The first sample was taken immediately after adding dissolution medium. This data is defined as zero-time point. The interval for each sample is 5 minutes. Each experiment lasts 45 minutes.

Analysis was carried out using an UV-visible spectrophotometer (Varian CARY 50 Bio) and 1-cm quarts cells at specified wavelengths, 242 nm for prednisone tablets and 296 nm for salicylic acid tablets (the approximate wavelength of maximum absorbance). The results were compared with a solution of known concentration of USP Reference Standard (RS). Experiments were performed six times in each location. Reference standard solutions for each drug were prepared in the dissolution medium of choice to generate an absorbance versus concentration standard curve. For prednisone tablet, a six-point calibration curve was obtained FIG. 6. The calibration curve has a linear equation with a slope of 0.0261 and interception of −0.0013. The R-squared value is 0.999. The concentration of the calibration curve ranged from 0.0007 mg/ml to 0.05 mg/ml. For salicylic acid tablet, a four-point calibration curve was plotted to get the concentration FIG. 7. The calibration curve has a linear equation with a slope of 0.0427 and interception of 0.0003. The R-squared value is 0.9988. The calibration curve ranged from 0.01 mg/ml to 0.1 mg/ml. For those concentrations that were not located inside the range, the sample would be diluted 10 times in order to make the concentration within the calibration range.

As discussed above, an embodiment of the present invention operates at an agitation speed of 35 rpm which mimics current USP classification system data for dissolution tests occurring at 50 rpm. A further embodiment of the present invention has an agitation speed of 66 rpm which mimics current USP classification system data for dissolution tests occurring at 100 rpm. Comparison of dissolution profiles between the present invention and current USP system can be found in S. Parekh, “Dissolution of Disintegrating Solid Dosage Forms in a Modified USP Dissolution Testing Apparatus 2,” Master's Thesis, NJ Inst. Tech., May 2011; X. Wu, “Dissolution Testing of Salicylic Acid Calibrator Tablets at Different Tablet Locations,” Master's Thesis, NJ Inst. Tech, May 2011 herein incorporated by reference. For any other dissolution speeds espoused by USP, the methods employed therein could be utilized to find the related agitation speed of the present invention to most closely mimic current dissolution profiles, therefore the present invention is able to mimic any current dissolution profile.

Certain experimental results are shown (FIG. 8 [USP system] and in FIG. 9 [one embodiment of inventive system]). Said release data showed a very consistent and reproducible trend even the tablets were in different locations when compared to the USP system.

In FIG. 10, nine dissolution profiles were plotted. Although the tablets were located at nine different locations during the implementation of one exemplary embodiment of the present invention, the release profiles were almost the same. They all have a very similar release pattern which indicated that the position of the tablet did not affect the dissolution results.

In certain embodiments of the present invention, the release data shows a very consistent and reproducible trend regardless of tablet location when compared to the USP system.

The dissolution profiles obtained from dosage forms are typically compared using two statistical approaches. The first approach consists of using a model-independent method based on the similarity factor (f₁) and difference factor (f₂) used by the FDA and originally proposed by Moore and Flanner:

$$\begin{matrix}{f}_{1} = \{ \frac{{\sum }_{t = 1}^{n} \vert {R}_{t} - {T}_{t} \vert}{{\sum }_{t = 1}^{n} {R}_{t}} \} \times 100 & ( 1 ) \\ {f}_{2} = 50 \cdot {log}_{10} \{ {( 1 + \frac{1}{n} {\sum }_{t = 1}^{n} {( {R}_{t} - {T}_{t} )}^{2} ]}^{- 0.5} \times 100 \} & ( 2 )\end{matrix}$$

where Rₜ is the reference assay at time t, Tₜ is the test assay at the same time, and n is the number of points. The higher the similarity factor f₁ (which can be in the range 0 to 100), the higher the average difference between reference and test curves is. The higher the difference factor f₂, (which can be in the range −∞ to 100) the lower the average difference between reference and test curves is. Public standards have been set by Food and Drug Administration (FDA) for f₁ and f₂. Accordingly, statistical similarity between the two curves being compared requires that both 0<f₁<15 and 50<f₂<100.

In order to have a more accurate and quantitative comparison, difference factor and similarity factor were obtained for certain embodiments of the present invention and a standard USP 2 device, as shown in Table 2. Position 1 where the center point located was treated as the reference test. For the exemplary embodiment utilized for testing, the maximum of the similarity factor was 78.8 and the minimum was 63.0. All the similarity factors were located in the range of 50-100, which indicated that the test release profiles were statistically similar to the reference release profile for said embodiment, according to the FDA guidelines. The difference factor ranged from 3.1 to 5.1, which showed a very low difference between the test release profile and the reference release profile for said embodiment. Both the similarity factor and difference factor ensured statistically similar curves between two release profiles for said exemplary embodiment, according to the FDA guidelines. The same is not true for the current USP 2 Apparatus.

Table 2 illustrates statistical evaluation of similarity between dissolution profiles of off-center tablets and centrally located tablets for Prednisone tablets at different locations with different statistical methods for different systems. Statistical similarity between the two curves being compared requires that both 0<f₁<15 (the lower, the better) and 50<f₂<100 (the higher, the better). Gray boxes indicate a failing value according to FDA criteria.

TABLE 2
Similarity factor and difference factor for prednisone tablets in standard
Apparatus 2 system (Bai and Armenante, 2009) Prior Art

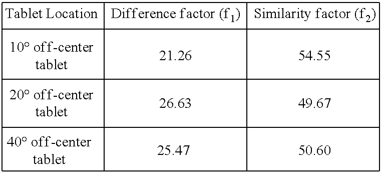

Similarity factor and difference factor for prednisone tablets in one
embodiment of the present invention

| Position # | Difference factor (f₁) | Similarity factor (f₂) |
|---|---|---|
| 2 | 5.1 | 63.0 |
| 3 | 4.3 | 72.2 |
| 4 | 3.3 | 78.8 |
| 5 | 3.1 | 74.4 |
| 6 | 3.9 | 66.0 |
| 7 | 3.3 | 74.6 |
| 8 | 3.1 | 71.1 |
| 9 | 3.7 | 75.6 |

Testing with one exemplary embodiment of the present invention also took place with salicylic acid. The results were shown in FIG. 10 (USP system) and in FIG. 11 (one embodiment of inventive system). In one embodiment of inventive system, the release data showed a very consistent and reproducible trend even when the tablets were in different locations when compared to the USP system.

In FIG. 11, nine dissolution profiles were plotted together in order to have a very clear comparison with each other for one embodiment of the present invention. Although the tablets were located at nine different locations, the release profiles were almost identical. They all have a very similar release pattern which indicated that the position of the tablet did not affect the dissolution results. Release data for said embodiment showed a very consistent and reproducible trend even the tablets were in different locations which is not the case with the USP system. FIG. 12 shows the positions mentioned in table 2.

In order to have a clear comparison between these release profiles, similarity factor f₂ and difference factor f₁ were calculated (Table 3) followed the method by Moore and Flanner (R) for certain exemplary embodiments of the present invention. Position 1 where the center point located was treated as the reference test. The maximum of the similarity factor was 88.0 and the minimum was 67.5 for one exemplary embodiment of the present invention. All the similarity factors were located in the range of 50-100, which indicated that the test release profiles were statistically similar to the reference release profile, according to the FDA guidelines. The difference factor ranged from 4.7 to 14.7, which showed a low difference between the test release profile and the reference release profile for said exemplary embodiment. All the difference factors were located in the range of 0-15, which indicated that the test release profiles were statistically similar to the reference release profile, according to the FDA guidelines.

Again, Table 3 illustrates statistical evaluation of similarity between dissolution profiles of off-center tablets and centrally located tablets for salicylic acid tablets at different locations with different statistical methods for different systems. Statistical similarity between the two curves being compared requires that both 0<f₁<15 (the lower, the better) and 50<f₂<100 (the higher, the better). Gray boxes indicate a failing value according to FDA criteria.

TABLE 3
Similarity factor and difference factor for salicylic acid in standard
Apparatus 2 system (Bai and Armenante, 2009) Prior Art

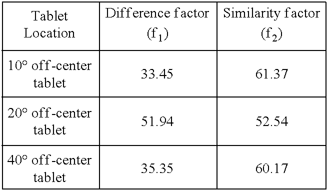

Similarity factor and difference factor for salicylic acid tablets for
one embodiment of the present invention

| Position # | Difference factor (f₁) | Similarity factor (f₂) |
|---|---|---|
| 2 | 12.2 | 71.7 |
| 3 | 4.7 | 88.0 |
| 4 | 11.4 | 72.8 |
| 5 | 8.5 | 78.5 |
| 6 | 14.2 | 68.2 |
| 7 | 14.7 | 68.7 |
| 8 | 11.7 | 71.4 |
| 9 | 14.9 | 67.5 |

In summary, two kinds of dosage forms were utilized in conducting dissolution testing for certain embodiments of the present invention. For both the prednisone calibrator tablet, a representative of a disintegrating tablet, and the salicylic acid calibrator tablet, a representative of a non-disintegrating tablet, robust and reliable release profiles were observed regardless of tablet location. The release profiles indicated a very robust and consistent release pattern for both dosage forms when compared to the current USP 2 Apparatus.

All of the similarity factors f₂ for said exemplary embodiments are located in range of 50 to 100, which implied that the release profiles at positions 2 to 9 are very close to that at position 1, the reference position, much closer in fact, than current USP 2 systems. Therefore, it is obvious that the dissolution rates measured for certain embodiments of the present invention are independent of the locations of tablet, thus solving a long-felt need in the art with respect to tablet-placement induced variability.

Although the systems and methods of the present disclosure have been described with reference to exemplary embodiments thereof, the present disclosure is not limited thereby. Indeed, the exemplary embodiments are implementations of the disclosed systems and methods are provided for illustrative and non-limitative purposes. Changes, modifications, enhancements and/or refinements to the disclosed systems and methods may be made without departing from the spirit or scope of the present disclosure. Accordingly, such changes, modifications, enhancements and/or refinements are encompassed within the scope of the present invention.

We claim:

1. An apparatus for dissolution testing, comprising:
a. a cylindrical, fluid filled vessel;
b. a stirring element placed below the liquid air interface of said vessel;
c. said stirring element positioned above the bottom of the vessel to allow for a dosage form to move freely; and
d. said stirring element located between about 0.1 mm to about 13 mm from center point of vessel bottom on the horizontal axis.

2. The apparatus as in claim 1, further comprising:
a. said stirring element located between about 2 mm to about 13 mm from center point of vessel bottom on the horizontal axis.

3. The apparatus as in claim 1, further comprising a means for adjusting the horizontal position of the stirring element in relation to the vessel.

4. The apparatus as in claim 3, wherein the means for adjusting the horizontal position of the stirring element in relation to the vessel comprises:
a. a vessel holding plate positioned perpendicularly to said stirring element;
b. said vessel holding plate comprising a plurality of circular cavities;
c. said cavities securing a vessel;
d. said cavities further comprising a plurality of removable spring and screw assemblies with at least one assembly removed and replaced with a pad; and
e. said assemblies tightened against said vessels such that the stirring element position is located between about 0.1 mm to about 13 mm from center point of vessel bottom on the horizontal axis.

5. The apparatus as in claim 4, further comprising:
a. said plurality of assemblies tightened such that the stirring element position is located between about 2 mm to about 13 mm from center point of vessel bottom on the horizontal axis.

6. The apparatus as in claim 3, wherein the means for adjusting the horizontal position of the stirring element in relation to the vessel comprises:
a. a circular insert of asymmetric thickness;
b. said insert secured within top lip of a vessel cavity;
c. said insert placed within said cavity securing a vessel; and
d. said asymmetric thickness imparting a horizontal shift in position such that the stirring element position is located between about 0.1 mm to about 13 mm from center point of vessel bottom on the horizontal axis.

7. The apparatus as in claim 6, further comprising:
a. said asymmetric thickness imparting a horizontal shift in position such that the stirring element position is located between about 2 mm to about 13 mm from center point of vessel bottom on the horizontal axis.

8. The apparatus as in claim 4, wherein the means for adjustment comprises:
a. a vessel holding plate positioned perpendicularly to said stirring element;
b. said vessel holding plate comprising a plurality of circular cavities;
c. said cavities securing a vessel;
d. a lever;
e. said lever activated such that lever movement effectuates a horizontal shift in said holding plate position;
f. said shift in holding plate position causing a horizontal shift in position such that the stirring element position is located between about 0.1 mm to about 13 mm from center point of vessel bottom on the horizontal axis

9. The apparatus as in claim 8, further comprising:
a. said shift in holding plate position causing a horizontal shift in position such that the stirring element position is located between about 2 mm to about 13 mm from center point of vessel bottom on the horizontal axis

10. The apparatus as in claim 9, further comprising:
a. A lever capable of multiple lever movement positions corresponding to multiple fixed shifts in holding plate position located within the range of said horizontal shift in position.

11. The apparatus as in claim 4, wherein the means for adjustment comprises:
a. a motor holding assembly;
b. a plurality of motor drives attached to said assembly;
c. one stirring element affixed to each motor drive;
d. a lever connected to said motor holding assembly;
e. said lever activated such that lever movement effectuates a horizontal shift in said motor holding assembly; and
f. said shift in motor holding assembly causing a horizontal shift in position of the stirring element of between about 0.1 mm to about 13 mm.

12. The apparatus as in claim 11, further comprising:
a. said shift in motor holding assembly causing a horizontal shift in position of the stirring element of between about 2 mm to about 13 mm.

13. A method for dissolution testing comprising:
a. filling a vessel with between about 300 ml and about 1000 ml of dissolution medium;
b. raising the temperature of the dissolution medium;
c. adjusting the vessel location such that a stirring element is off-center with respect to vessel bottom when inserted vertically from above;
d. depositing a dosage form within the vessel;
e. inserting the stirring element in the vessel such that the element is within the vessel and the entire stirring element is under the liquid air interface;
f. engaging the stirring element within the testing solution at an agitation speed between 25 rpm and 200 rpm;
g. disengaging the stirring element after a period of about 15 minutes to about 180 minutes; and
h. measuring the absorbance versus concentration.

14. The method according to claim 13, further comprising:
a. adjusting the vessel location such that the stirring element position is located between about 0.1 mm to about 13 mm from center point of vessel bottom on the horizontal axis.

15. The method according to claim 13, further comprising:
a. adjusting the vessel location such that the stirring element position is located between about 2 mm to about 13 mm from center point of vessel bottom on the horizontal axis.
